(19) United States
(12) Patent Application Publication
(10) Pub. No.: US 2012/0045814 A1
(43) Pub. Date: Feb. 23, 2012

(54) Method of Producing Synthetic Petroleum From Coal or Hydrocarbons or From C,H or Oxygen Using a Host Microorganism

(76) Inventor: Mrugesh Shah, (US)

(21) Appl. No.: 13/286,304

(22) Filed: Nov. 1, 2011

Related U.S. Application Data
(63) Continuation of application No. 10/776,711, filed on Feb. 12, 2004, now abandoned.
(60) Provisional application No. 60/447,204, filed on Feb. 13, 2003.
(60) Provisional application No. 60/462,377, filed on Apr. 11, 2003.

(51) Int. Cl. C12P 1/04

(52) U.S. Cl. 435/170

(57) ABSTRACT

The invention relates to a method of making microorganisms capable of producing petroleum from coal, or wood or certain other fossil fuels or raw materials, wherein gene sequences responsible for such production are isolated from microorganisms capable of such production, and transfected into suitable hosts, with better productivity or viability. The invention also includes using the same process to make elemental carbon, hydrogen and oxygen from organic or inorganic sources, including natural water or salt-water sources, petroleum, coal, other fossil fuel materials or other hydrocarbon sources, including turf, grass, glucose, rubber, sapropel, sapropelites, slates and wood; and it further includes making fossil fuels from water or from carbon, hydrogen and oxygen. In the alternative, the appropriate gene sequence can be used to make probes which can be used to find other gene sequences in other microorganisms which can optimize production.

### RELATED APPLICATIONS

This application claims priority to U.S. Provisional application Nos. 60/447,204, filed on Feb. 13, 2003, and 60/462,377, filed on Apr. 11, 2003.

### BACKGROUND OF THE INVENTION

Certain microorganisms can produce petroleum from solid fossil fuels, including coal, as well as from oil tars obtained by distillation of coal, turf, grass, glucose, rubber, sapropel, sapropelites, slates, wood and other raw materials. See International Patent Application No. WO 0246446, describing the conditions of production. Other microorganisms are capable of converting glucose, rubber and other organic material into petroleum. Genetic engineering techniques, i.e., transfection of the applicable genes into a host microorganism which has preferred characteristics, can be used to generate microorganisms which can effect the conversion process more efficiently. In addition to the microorganisms described in the above-noted international patent application, which are specific strains of Thiobacillus aquaesullis, Thiosphaera pantotropha (also known as Paracoccus pantotrophus, deposited at the American Type Culture Collection, Manassas Va., under Accession No. 35512; and also described in: Robertson and Kuenen, Int. J. Syst. Bacteriol. 49:650 (9184)) and Thoibacillu thoioparus (which is used only when the raw material has a pH equal to or less than 5.5), other microorganism strains exist in nature which can be optimal hosts, or which themselves can efficiently carry out the conversion process. For example, other microorganisms, or other strains, including, for example, those that exist in water, and especially in deep water, could be explored for their ability to perform the conversion. Such microorganisms tend to grow and reproduce more quickly, and be more productive metabolically, due to the highly nutritious environment they are in, with highly compressed nitrates, carbon dioxide, carbon monoxide, and decomposition gases such as methane, phosphates and oxygen available to be metabolized. The genes from such microorganisms can be isolated and used to make a genetically engineered host with optimal characteristics.

### SUMMARY OF THE INVENTION

The invention relates to a method of making microorganisms capable of producing petroleum from coal, or wood or certain other fossil fuels, or raw materials including turf, grass, glucose, rubber, sapropel, sapropelites, slates and wood, in a highly efficient, commercially viable manner. The method involves isolating gene sequences responsible for such production from microorganisms capable of such production, then transfecting these gene sequences into other host cells, including plant cells such as algae, or into other microorganisms which reproduce or grow more quickly, to produce more petroleum per unit organism or per unit nutrient or raw material. Those with desired characteristics can be further selected, to find those which can produce the petroleum in an optimal commercially viable manner. The invention, and the manner of making and using it, is described further below.

The invention also includes the making of elemental carbon, hydrogen and oxygen from organic or inorganic sources, including natural water or salt-water sources, petroleum, coal, other fossil fuel materials or other hydrocarbon sources, including turf, grass, glucose, rubber, sapropel, sapropelites, slates and wood. This can be accomplished using natural or recombinant bacteria or organisms which convert hydrocarbons into these elements. Again, genes from naturally-occuring bacteria which accomplish this conversion can be transfected to other hosts to optimize production.

The invention also includes the making of hydrocarbons, including petroleum, from water or from elemental carbon, hydrogen and oxygen. This can be accomplished using natural or recombinant bacteria or organisms which convert these elements into hydrocarbons. Again, genes from naturally-occuring bacteria which accomplish this conversion can be transfected to other hosts to optimize production.

It is noted that the transfection steps described above can be accomplished with viral vectors (e.g., adenovirus) or other vectors or plasmids. Nanotechnology instrumentation can be used to manipulate the viral vectors for the transfection.

### DESCRIPTION OF THE DRAWINGS

FIG. 1 is a schematic depiction of a subtractive hybridization process, starting with a tester and a driver microorganism, wherein the tester has the target gene sequence of interest (depicted as a dotted line). The first step, (arrow I.) is to isolate RNA from the respective microorganisms. Step II. is to from cDNA from the tester RNA and labeled cDNA from the driver RNA. Step III. is to hybridize the respective cDNAs, and then to cleave them with restriction endonucleases. Step IV. is to separate the labeled annealed fragments and the target gene of interest (dotted line). The target gene can then be amplified with PCR.

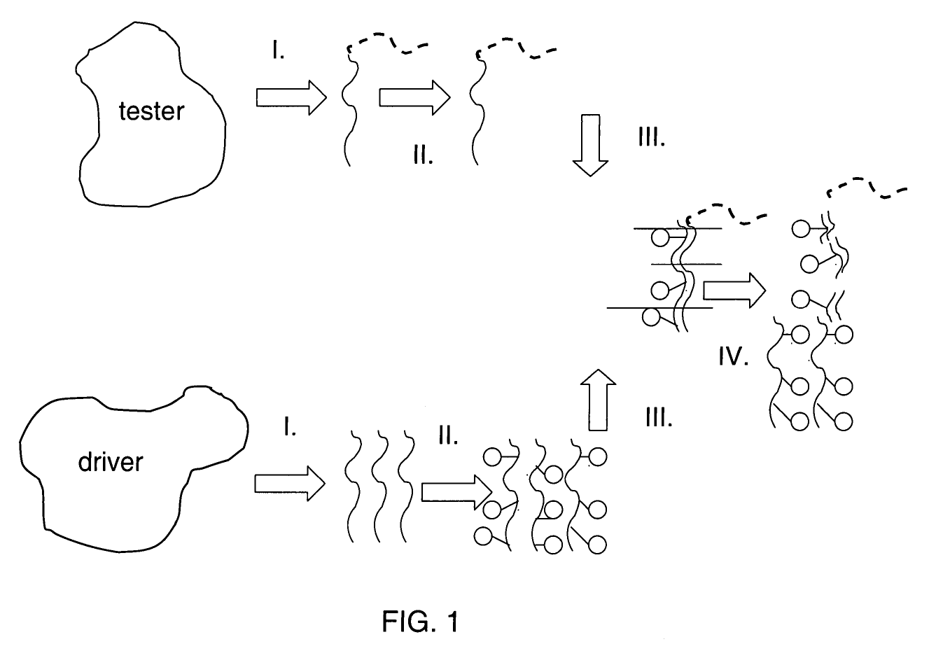

### DETAILED DESCRIPTION OF THE INVENTION

Among the techniques for isolating the gene sequences responsible for a particular function is subtractive hybridization. Subtractive hybridization allows one to enrich for nucleic acid sequences present in one sample but absent, decreased, or altered in another sample. See O. D. Ermolaeva et al., Genetic Anal.: Biomol. Eng. 13:49-58 (1996). A “target” in such methods is a gene or set of nucleic acid sequences to be enriched, and the “tester and driver” are the nearly identical nucleic acid samples that preferably differ from one another only by the presence or absence of the target sequence(s). In the case of using this method to isolate genes capable of producing petroleum from fossil fuels, one could use the bacterial strains capable of such production, e.g., Thiobacillus aquaesullis 4255 and 389, Thiosphaera pantotropha 356, Thiosphaera pantotropha 2944, and Thoibacillu thoioparus 55, or mutations or variant strains, to provide the tester sequences (all of which are described in International Patent Application No. WO 0246446), and other strains of the bacteria Thiobacillus aquaesullis, Thiosphaera pantotropha and Thoibacillu thoioparus which do not have this ability, to provide the driver sequences. In the alternative, one could use the genes from other bacterial strains which are capable of producing petroleum from raw materials to provide a tester sequence, and use a related strain to provide the driver.

Following extraction of the driver and tester RNA, cDNA is prepared therefrom. Using cDNA is preferred over use of genomic DNA, because the RNA only includes exons, or coding portions of the gene, and not the non-coding introns. The driver DNA and tester DNA are fragmented, using restriction enzymes, and the driver is labeled to enable subsequent purification. Finally, a mixture of the fragmented DNAs, in which driver is in substantial excess over tester, is heat denatured and complementary single strands are allowed to re-anneal. Due to the excess of driver over tester, a majority of tester sequences with sequences common to the driver sequences will hybridize with the drivers. This hybridization will allow one to eliminate the sequences common to driver and tester, because the driver is labeled for identification in the population. The only species left will be sequences of the tester which do not have a corresponding driver sequence, which includes mainly those including target sequences. If further enrichment is required, additional rounds of subtraction are performed. Finally, individual fragments cloned from the subtraction products can be amplified using polymerase chain reaction (see U.S. Pat. No. 4,683,195; Saiki et al., Science 230:1350-1354 (1985)), which can then be more easily screened for target sequences, which in this case would be those sequences related to conversion of fossil fuels and other raw materials to petroleum.

Representational Difference Analysis (RDA) is a related method of subtractive hybridization that incorporates polymerase chain reaction (PCR) as an integral part of the procedure. The success of PCR-based subtractive hybridization is partially dependent on the initial amplicon complexity and/or the relative abundance of target sequence within an amplicon. An amplicon includes the set of nucleic acid sequences amplified by PCR. If the complexity is too high, or if the target sequence concentration is too low, the kinetics of hybridization prevent effective enrichment, and the method fails. Following the amplification, the target sequences are subject to subtractive hybridization using an excess of driver sequences as described above.

Amplicon complexity is reduced in the RDA procedure by the amplification of only a representative subset of all possible fragments from driver and tester. Such subsets are achieved by selective amplification of nucleic acid fragments based on their size, such that only those of a certain size are amplified. Alternatively, the starting nucleic acid can be enriched for target sequences prior to subtraction by partial purification, accomplished by passing the sample through a two-micron filter prior to extraction, thereby eliminating most of the cellular nucleic acids present in the sample and alleviating the necessity of reducing amplicon complexity. See Simons et al., Proc. Natl. Acad. Sci. USA 92:3401-3405 (1995). The RDA procedure can also be used to select for the nucleic acid sequences coding for petroleum production in the strains Thiobacillus aquaesullis 4255 and 389, Thiosphaera pantotropha 356, Thiosphaera pantotropha 2944, and Thiobacillu thoioparus 55, or mutations or variant strains.

Following isolation of target sequences using subtractive hybridization, they could also be directly transfected into a host microorganism, using conventional techniques, to attempt to produce a microorganism capable of producing petroleum from solid fossil fuels in a highly efficient, commercially viable manner. The transfection can be done using viral vectors or plasmids, following conventional procedures. Following transfection, a number of new candidate microorganisms are produced, which include the genes of interest. These new recombinant microorganisms are then tested to attempt to isolate the ones which are capable of production of petroleum with optimal efficiency. Microorganisms suitable as hosts include those inhabiting salt water or fresh water, stagnant water, water which is chemically altered; those capable of metabolizing glucose and other conventional nutrient media, those inhabiting rocky, sandy or sand/water environments, those capable of surviving heat, cold, or acidic or basic environments, those oxidizing sulfur, and aerobic and anaerobic bacteria. The host microorganism best suited for commercial production can have some or several of these characteristics, depending on how it is to be cultured. As noted above, preferred host microorganisms include those which inhabit water, including deep water. Such microorganisms are generally capable of growing more quickly, due to their nutrient-rich environment.

Following isolation of target sequences using subtractive hybridization, they can, in the alternative or in addition to direct transfection into hosts, be used to make oligonucleotide probes, which are complementary to target sequences and which can be used to “fish out” sequences which are homologous to target sequences in other microorganism strains. For example, one could examine other microorganisms which have ability to convert raw materials to petroleum. The probes can be spotted in an array and contacted with amplicons (amplified by PCR) from genomic DNA from the microorganisms of interest under hybridizing conditions. See, e.g., WO03/034029 (Background Section). In such case, the primers for the amplification would be designed based on the known sequence of the probe terminal regions. The amplicons which hybridize to the probes can be transfected into hosts, and it can be determined if such transfected hosts are more efficient in conversion, or otherwise better suited for production. This process, therefore, permits the optimization of the sequences for production.

It is likely that there are a number of microorganisms existing in water, especially in deep water, which already have the genes and the capability of converting fossil fuels, or other nutrient media including animal or vegetable matter, into petroleum. It is also likely that there are microorganisms which can generate elemental carbon, hydrogen and oxygen from organic or inorganic sources, including natural water or salt-water sources, petroleum, coal, other fossil fuel materials or other hydrocarbon sources, including turf, grass, glucose, rubber, sapropel, sapropelites, slates and wood. An alternative to starting with the exemplary microorganisms set forth above is to test microorganisms from water for such ability, and then isolating the genes using the techniques described above. The genes can be transfected into a host microorganisms which has desired characteristics, such as faster growth, reproduction, higher productivity or the ability to survive adverse production conditions.

An alternative method of attempting to improve the production characteristics of the microorganism is by selective alteration of the sequence of the gene responsible for the conversion ability. Once this gene is isolated and sequenced, it can be modified in a selective step-wise fashion, such as replacement of one base at a time, and the resulting gene can then be transfected into a host and tested for its conversion ability. This method can lead to further improvements in growth, reproduction, or survivability of the host, as well as optimal efficiency and productivity.

The foregoing terms and expressions are not intended to be limiting, but are exemplary only, and the scope of the invention is defined only in the claims that follow, and includes all equivalents of the subject matter of those claims. The methods described herein are not limited to the order of steps set forth, and include any order of steps.

1.-3. (canceled)

4. A method of converting (i) solid fossil fuels, or (ii) oil tars obtained by distillation of coal, turf, grass, rubber, sapropel, sapropelites, slates, or wood, into biosynthetic petroleum, comprising the steps of:
a) isolating a starting microorganism capable of said conversion;
b) isolating from the starting microorganism the genes responsible for the conversion ability;
c) transfecting the genes into a host microorganism, and
d) combining the host microorganism with the solid fossil fuels or oil tars under conditions suitable for the conversion of the solid fossil fuels or oil tars into biosynthetic petroleum.

5. The method of claim 4, wherein the starting microorganism is Thiobacillus aquaesulis 4255 or 389, Thiosphaera pantotropha 356, Thiosphaera pantotropha 2944, Thoibacillus thoioparus 55, or mutants or variants thereof, or a microorganism which exists naturally in water.

6. A method of improving conversion of (i) solid fossil fuels, or (ii) oil tars obtained by distillation of coal, turf, grass, rubber, sapropel, sapropelites, slates, or wood, into biosynthetic petroleum, comprising the steps of:
(a) isolating a starting microorganism capable of said conversion;
(b) isolating from the starting microorganism an oligonucleotide probe complementary to a gene responsible for the conversion ability;
(c) placing the probe under hybridizing conditions in contact with amplicons from other microorganisms suspected to be capable of or being capable of said conversion;
(d) isolating amplicons which hybridized;
(e) transfecting the isolated amplicons into a host microorganism;
f) combining the host microorganism with the solid fossil fuels or oil tars under conditions suitable for the conversion of the solid fossil fuels or oil tars into biosynthetic petroleum; and
(g) determining whether productivity improved.

7. A method of converting carbon, hydrogen and oxygen into-biosynthetic coal or biosynthetic petroleum, comprising the steps of:
(a) isolating a starting microorganism capable of said conversion;
(b) isolating from the starting microorganism the genes responsible for the conversion ability;
(c) transfecting the genes into a host microorganism; and
(d) combining the host microorganism with the carbon, hydrogen and oxygen under conditions suitable for the conversion of the carbon, hydrogen and oxygen into biosynthetic coal or biosynthetic petroleum.

8. The method of claim 7 wherein, after transfection, the transfected host microorganism as compared to the starting microorganism is capable of faster growth, reproduction, enhanced survivability in a production environment, or more production of biosynthetic coal or biosynthetic petroleum per unit of a nutrient.

9. The method of claim 7 wherein the host microorganism can exist in salt water or fresh water, can metabolize glucose, rubber, grass, or other nutrient media, can survive acidic or basic environments, can oxidize sulfur, or can exist in aerobic or anaerobic conditions.

10. The method of claim 7 wherein the genes responsible for conversion are isolated by subtractive hybridization.

11. The method of claim 10 wherein the subtractive hybridization is performed by representational difference analysis.

12. A method of converting (i) solid fossil fuels, or
(ii) oil tars obtained by distillation of coal, turf, grass, rubber, sapropel, sapropelites, slates, or wood,
into biosynthetic petroleum, comprising the steps of:
a) obtaining a gene encoding a protein capable of said conversion;
b) transfecting the gene into a host microorganism, and
c) combining the host microorganism with the solid fossil fuels or oil tars under conditions suitable for the conversion of the solid fossil fuels or oil tars into biosynthetic petroleum.

13. A method of converting carbon, hydrogen and oxygen into biosynthetic coal or biosynthetic petroleum, comprising the steps of:
a) obtaining a gene encoding a protein capable of said conversion;
b) transfecting the gene into a host microorganism; and
c) combining the host microorganism with the carbon, hydrogen and oxygen under conditions suitable for the conversion of the carbon, hydrogen and oxygen into biosynthetic coal or biosynthetic petroleum.
